(19) United States
(12) Patent Application Publication
(10) Pub. No.: US 2009/0299337 A1
(43) Pub. Date: Dec. 3, 2009

(54) SELF-LOCKING FLUID CONNECTION

(75) Inventors: Christopher J. GROPPI, Franklin, WI (US); Dean A. Erickson, Greenfield, WI (US); Robin Fitzgerald, St. Francis, WI (US)

Correspondence Address: SWANSON & BRATSCHUN, L.L.C., 8210 SOUTHPARK TERRACE, LITTLETON, CO, 80120, US

(73) Assignee: BioForm Medical, Inc.

(21) Appl. No.: 12/131,659

(22) Filed: Jun. 2, 2008

(51) Int. Cl. A61M 39/10

(52) U.S. Cl. 604/533; 604/264

(57) ABSTRACT

A self locking luer type connection. The luer lock includes a male and female luer portion adapted to removably connect to each other and capable of forming a sealed fluid flow path. A combination of both hard and semi-hard materials is utilized for either a male luer portion or a female luer portion to create a sealing surface that features the benefits of both materials. By applying a less rigid material to a rigid base, the luer lock connection is able to be used multiple times, have a large range of tightening positions that function to create a seal, will be reliable in high-pressure connections, and will function with multiple types of the corresponding luer portion.

### FIELD OF THE INVENTION

The present invention relates generally to the field of fluid connectors. Specifically, the present invention relates to luer connections for syringes.

### BACKGROUND OF THE INVENTION

Luer connections are utilized in many different fields. Standard male syringe luers are comprised of uniform rigid or semi-rigid materials to create a sealing surface with female luers. This seal is formed through the tightening of the fluid path wall of the male luer taper to the fluid path wall of the female luer taper. The rigidity of the materials used to create the male and female luers affect the seal strength, the ability of the seal to be re-applied, and the tightening position at which this seal functions. Typically prior art luer locks have relied upon the fit of a rigid male member with a rigid female luer portion. Rigid luer locks provide for a relatively long useful life, but have a narrow “tightening zone” which is the range of tightness of the male and female luer that will maintain a fluid seal. As most prior art luer locks utilize a threaded connection between the male and female luer portions, the tightening zone is often expressed in terms of degrees of rotation, i.e. at how many degrees rotation are required before a seal is formed when threading the luer lock components together.

Other prior art devices have utilized semi-rigid, elastic components rather than rigid components. Elastic components provide for a larger tightening zone. However, elastic components suffer from a loss of elasticity over time, making them ill suited for repeated usage including, for example, serially connecting multiple syringe bodies to one needle or multiple needles to one syringe body.

The more rigid materials commonly provide better dimensional stability and ability to be re-used, but also have less reliable seals in high-pressure use connections and a smaller range of tightening positions that function when sealing. The less rigid materials commonly provide less dimensional stability and a lower ability to create a seal after multiple uses, but they are more reliable in single use high-pressure connections and have a larger range of tightening positions that function when sealing.

The combination of one rigid luer and one corresponding semi-rigid luer to achieve the benefits of both rigid and semi-rigid materials is not optimal for two reasons. First, the durometer that can be used for the semi-rigid luer is limited because structural strength must be maintained; i.e., a low durometer male or female luer portion may not withstand the necessary stresses. Second, the two luer types would have to be used in conjunction with each other in order to be effective. Therefore, interchangability would be limited as rigid/semi-rigid combinations would not be available with all versions of luers available.

### SUMMARY OF THE INVENTION

The present invention relates to fluid connectors, such as a luer lock type connector, that are easier to tighten and allow for more size/shape variation in the corresponding luer portions. One embodiment comprises a male luer member having a hard tubular member with a first end and a second end and with a fluid passage therethrough and also a semi hard sealing member that is disposed about at least a portion of the tubular member. The semi-hard sealing member comprises material having a durometer of about 10-100 Shore A. The hard tubular member comprises material having durometers of above about 50 Shore A with the hard tubular member further having at least about 10-20 Shore A greater durometer than the associated semi-hard sealing member. This correlation enables deformation to occur preferentially in the semi-hard sealing member for any given set of durometers. The connector further comprises a female luer member having a first and second end and a body with a fluid passage therethrough. The first end of the female luer member consists of a chamber integral with the fluid passage and adapted to receive the male luer member, wherein when the second end of the tubular member is inserted into the first end of the female luer member, the sealing member engages the chamber and provides a seal between the chamber and the tubular member forming a continuous fluid flow passage. The durometer of the reusable female luer member is at least about 10-20 Shore A greater durometer than the semi-hard male luer member. In one embodiment, the pressure seal is maintained at the tip of the male luer member and the base of the female luer member, thereby preventing any material separation at the locus of the seal joints.

In another embodiment, the present invention relates to a syringe assembly comprising a needle, a syringe and a connector there between. The needle has a fluid flow path therethrough, the needle affixed to a first end of a connection joint. The connection joint has a fluid flow path therethrough and connected at a second end to a fluid container. The connection joint comprises a first portion and a second portion, the first portion having a protrusion and a seal circumscribed about at least a portion of the protrusion; and the second portion has an opening for receiving the protrusion. The seal comprises material has a durometer of between about 10 and 100 Shore A. The first portion and second portion are adapted to be coupled such that a continuous fluid flow path is formed therethrough, with the protrusion partially disposed within the second portion through the opening and sealed by interaction of the seal against an inner wall of the second portion.

The invention includes certain features and combinations of parts hereinafter fully described, illustrated in the accompanying figures, described below, and particularly pointed out in the appended claims, it being understood that various changes in the details may be made without departing from the spirit, or sacrificing any of the advantages of the present invention.

### BRIEF DESCRIPTION OF THE DRAWINGS

FIG. 1 illustrates one embodiment of a luer lock connection;

FIG. 2 illustrates one embodiment of a female luer portion of the luer lock;

FIG. 3 illustrates one embodiment of a male portion of the luer lock;

FIG. 4 illustrates one embodiment of a sealing member on the male portion;

FIG. 5 illustrates another embodiment of a sealing member on the male portion;

FIG. 6 illustrates yet another embodiment of a sealing member on the male portion;

FIGS. 7A and 7B illustrate yet another embodiment of the sealing member, wherein the sealing member includes a tapered shape; and

FIG. 8 illustrates a needle and syringe assembly utilizing the luer lock of FIG. 1.

### DETAILED DESCRIPTION OF THE EXEMPLARY EMBODIMENTS

The present invention is directed to a luer lock type connector forming a connection joint. The luer lock involves the combination of two materials on one of either a male luer portion 103 or a female luer portion 105 to create a sealing surface that features the benefits of both materials. By applying a less rigid material to a rigid base, the luer lock 101 will be usable multiple times, have a large range of tightening positions that create an adequate seal, will be reliable in high-pressure connections, and will function with multiple types of the corresponding luer portion.

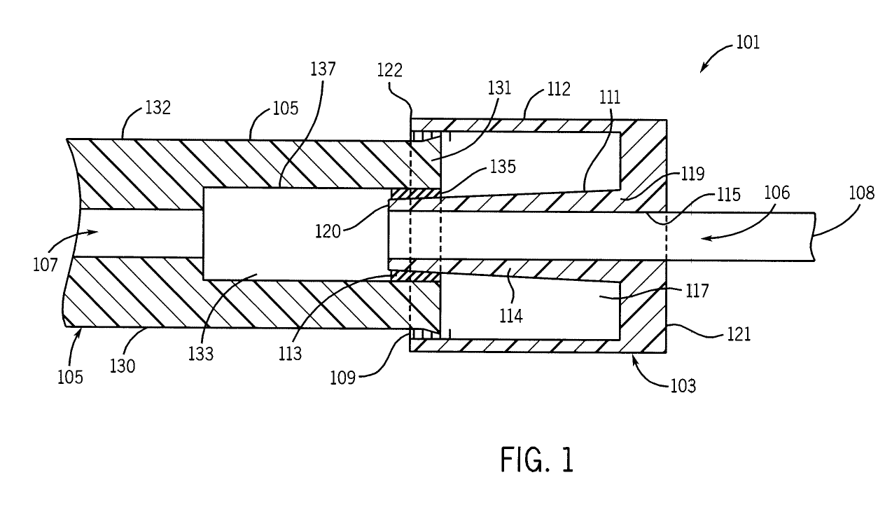

Referring to FIG. 1, the luer lock 101 includes a male luer portion 103 and a female luer portion 105. The male luer portion 103 has a male fluid flow path 106; and, likewise, the female luer portion 105 has a female fluid flow path 107. When the male luer portion 103 and the female luer portion 105 of the luer lock 101 are connected, as shown in FIG. 1, the male fluid flow path 106 and the female fluid flow path 107 form a continuous fluid flow path 108 through the luer lock 101.

The male luer portion 103 includes a inner male portion 111 and, in exemplary embodiments, on outer male portion 112. The inner male portion 111 has a first end 119 and a second end 120, with the second end proximate the female luer portion 105 when the respective male and female luer portions 103, 105 are connected. The inner male portion 111 includes a tubular member 114 through which the male fluid flow path 106 is positioned. The inner male portion 111 further includes a sealing member 113.

In one embodiment, the outer male portion 112 substantially surrounds the inner male portion 111, such that the inner male portion 111 is substantially disposed within the outer male portion 112. The positioning of the respective inner and outer male portions 111, 112 results in a male portion interstitial space 117 between the respective inner and outer portions 111, 112.

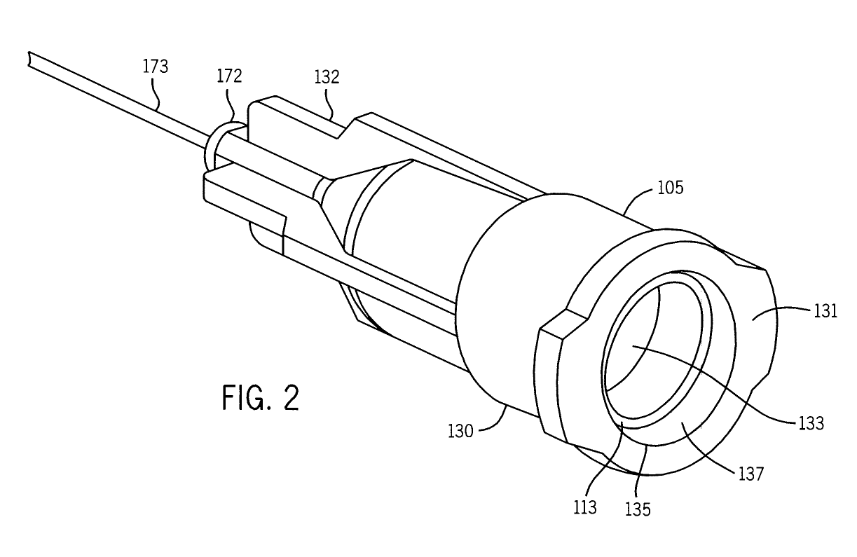

As shown in FIGS. 1 and 2, the outer male portion 112 is tubular having a outer male portion first end 121 and a outer male portion second end 122, oriented in the same manner as the respective first and second ends 119, 120 of the inner male portion 111. The outer male portion 112 includes a connection 115 for affixing the inner male portion 111 to the outer male portion 112. In an exemplary embodiment, the connection 115 forms a wall at the first end 121 of the outer male portion 112 with the male fluid flow path 106 passing through the wall and the inner male portion first end 119 being sealingly connected to the connection 115. In one embodiment, the male luer portion 103 comprises one unitary structure. Thus, the male luer portion 103 may be created using techniques such as various known molding processes and, for embodiments where the sealing member 113 is on the male luer portion 103, the sealing member 113 is then applied to the inner male luer portion 111.

As shown in FIGS. 1 and 2, the female luer portion 105 includes a body 130, in one embodiment a tubular body, having a first end 131 and a second end 132, with the first end 131 proximate the male luer portion 103 when the respective male and female luer portions 103, 105 are connected. The female fluid flow path 107 is disposed within the body 130 and passes there through. Thus, the female luer portion body 130 includes an aperture 135 at the first end 131. In an exemplary embodiment, the female luer portion body 130 includes a chamber 133 proximate the aperture 135 and forming an enlarged portion of the female fluid flow path 107. In one embodiment, the female luer portion 105 comprises one unitary structure. Thus, the female luer portion 105 may be created using techniques such as various known molding processes and, for embodiments where the sealing member 113 is on the female luer portion 105, the sealing member 113 is applied to the female luer chamber 133.

In use, the male luer portion 103 and the female luer portion 105 engage each other to form the continuous fluid flow path 108 (see FIG. 1). The first end 131 of the female luer portion body 130 is inserted into the male portion interstitial space 117 between the inner male portion 111 and the outer male portion 112. The inner male portion 111 is simultaneously inserted into the chamber 133 of the female luer portion 105.

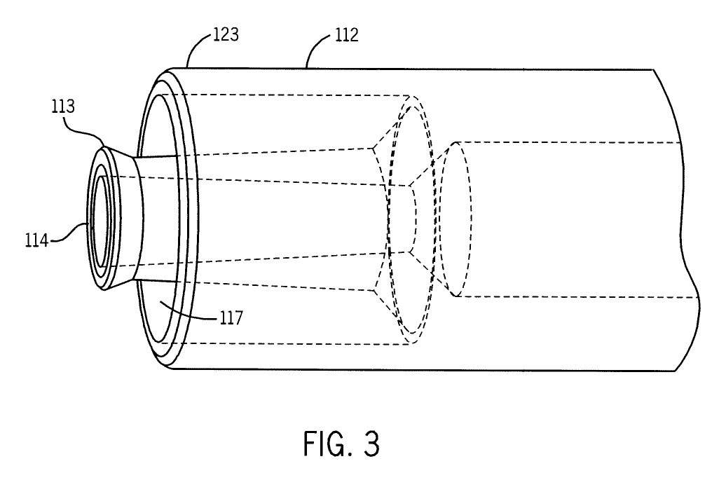

In one embodiment, either one or both of the male luer portion 103 and female luer portion 105 include a connection mechanism 109 for retaining the connection between the male luer portion 103 and the female luer portion 105 (see FIGS. 1-3). The connection mechanism 109 can include, but is not limited to, friction fit, threaded, latched, and snap-fit.

As seen in FIGS. 1, 2 and 4-7, sealing member 113 is positioned on the outer surface of the tubular member 114. It should be appreciated that the sealing member 113 could also be positioned on the inner diameter of the female luer portion 105 (FIG. 2). When the male luer portion 103 and the female luer portion 105 are connected (see FIG. 1), the sealing member 113 is positioned between the tubular member 114 and the inner wall 137 of the chamber 133. In one embodiment, the sealing member 113 can be integral with the tubular member 114, such as by injection molding. It will be appreciated that various method for positioning the sealing member 113 on the rigid tubular member 114 are known in the art, including, but not limited to, adhesive bonding, co-injection, overmolding, press-fit, friction fit, snap fit, ultrasonic weld, or other welding techniques. In an exemplary embodiment, the tubular member 114 is a rigid structure. In one embodiment, the rigidity of the “rigid” structures, such as the tubular member 114, is greater than about 50 Shore A. In one preferred embodiment, the rigidity of the “semi-rigid” structures, including the sealing member 113, is within the range of about 10 to about 100 Shore A. The tubular member 114 serves to support, along with the outer male portion 112, the connection against any stress that would bend the luer lock 101, potentially breaking the connection between the male luer portion 103 and the female luer portion 105.

In other embodiments, the male luer portion 103 and the female luer portion 105 are tubular. As such, the first end 131 of the female luer portion body 130 has an inner diameter and an outer diameter with a thickness of material there between. Likewise, the outer male portion 112 has an inner diameter and an outer diameter with a thickness of material there between. For the inner male portion 111, the tubular member 114 has an inner diameter and an outer diameter with a thickness of material there between; and the sealing member 113 has an inner diameter and an outer diameter with a thickness of material there between. In an exemplary embodiment as shown in FIG. 1, the inner diameter of the outer male portion 112 should be greater than the outer diameter of the first end 131 of the female luer portion 105, allowing the female luer portion 105 to be disposed between the outer male portion 112 and the inner male portion 111. Likewise, the inner diameter of the first end 131 of the female luer portion 105 is greater than the outer diameter of the tubular member 114, allowing the tubular member 114 to be disposed within the female luer portion 105. The diameter of the sealing member 113 is greater than the diameter of the tubular member 114; and in one embodiment is greater than the inner diameter of the first end 131 of the female luer portion 105 and in another embodiment less than that inner diameter. In cases where the sealing member 113 has a greater diameter than the inner diameter of the aperture, the sealing member 113 most preferably should be able to deform sufficiently to allow connection and sealing of the male luer portion 103 and the female luer portion 105.

In one embodiment, the sealing member 113 is positioned adjacent to the second end 120 of the inner male portion 111. The position at the second end 120, results in the sealing member 113 being in contact with the inner wall 137 of the chamber 133 substantially the entire time the male luer portion 103 and female luer portion 105 are in contact. In an alternative embodiment, the sealing member 113 is placed a distance from the second end 120.

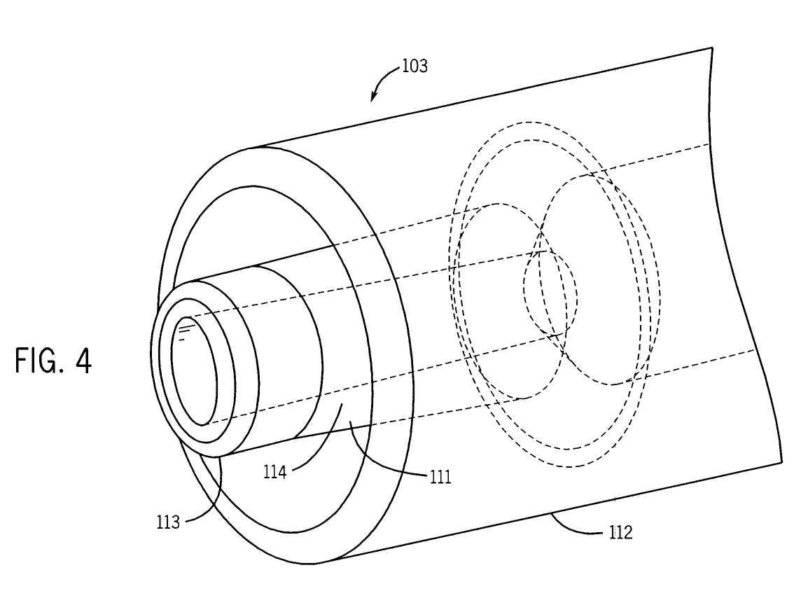

The sealing member 113 may be positioned at any point along the tube member 114 such that it is still capable of forming a seal between the male luer portion 103 and the female luer portion 105. In an exemplary embodiment, the sealing member 113 is disposed about the entire circumference of the tubular member 114. The sealing member 113 may extend all or only a portion of the length of the tubular member 114 (between the first end 119 and the second end 120). FIGS. 4-6 illustrate alternative embodiments of the sealing member 113.

In one embodiment, FIG. 4 illustrates the sealing member 113 having a substantially uniform thickness disposed about the circumference of the tubular member 114.

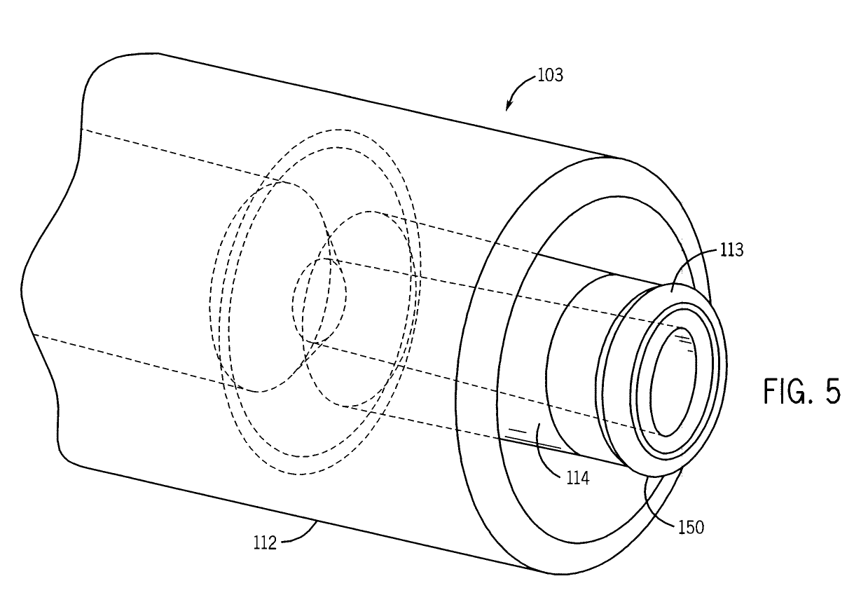

FIG. 5 illustrates an embodiment of the sealing member 113 wherein the sealing member 113 includes a single ridge 150 that is an area of greater thickness and that circumscribes the circumference of the tubular member 114 perpendicular to the longitudinal axis of the tubular member 114.

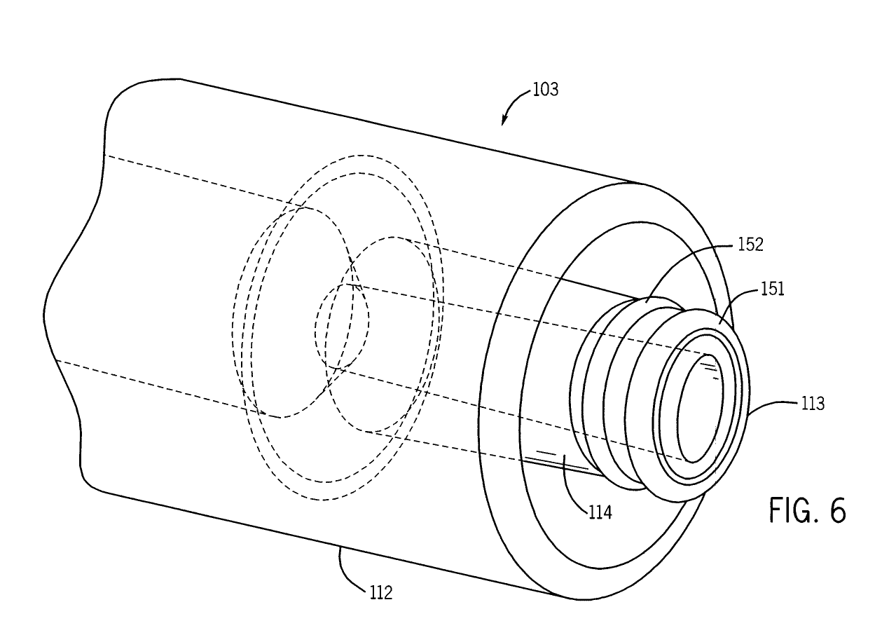

FIG. 6 illustrates yet another embodiment of the sealing member 113. The sealing member of FIG. 6 includes two ridges 150, an inner ridge 151 and an outer ridge 152 circumscribe the tubular member 114 substantially parallel to each other and perpendicular to the longitudinal axis of the tubular member 114.

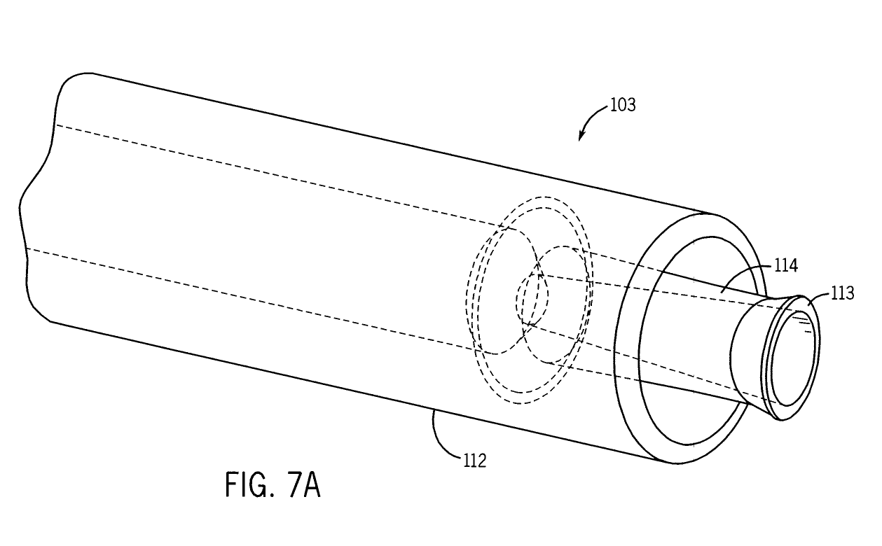

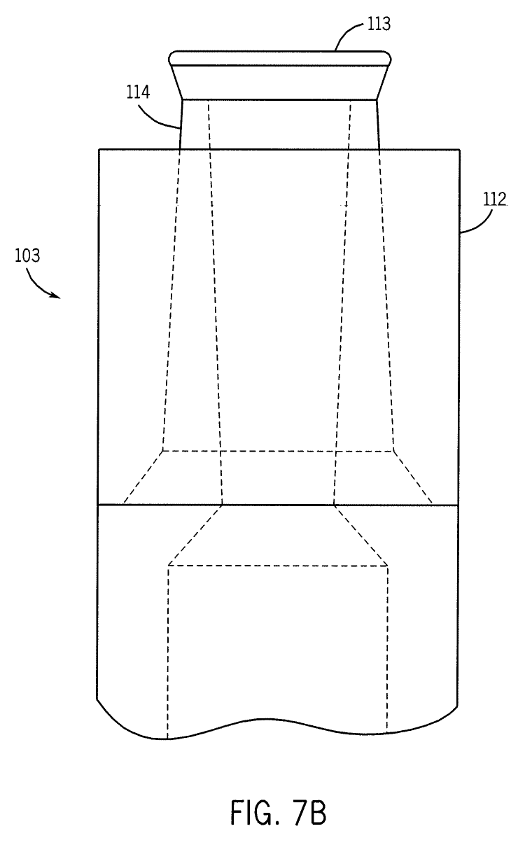

FIGS. 7A and 7B illustrate yet another embodiment of the sealing member 113, wherein the sealing member includes a tapered shape. It should be appreciated that the taper may slope away from the connection of the male portion 103 and female luer portion 105, as shown in FIGS. 7A and 7B, or towards that connection.

The sealing member 113 is comprised of a semi-rigid material. Such material may include, but is not limited to: thermoplastic or thermoset elastomers of Shore A durometers in the range of 10-100, including but not limited to thermoplastic or thermoset elastomers and include such materials as silicone, rubber, polyurethane, polyethylene, nylon, polyester, and polysulfone. The hardness or rigidity of the sealing member may be measured using a durometer. In an exemplary embodiment the sealing member exhibits a durometer of about 10-100 Shore A. In a further embodiment, the durometer is about 30-80 Shore A and in yet a further embodiment, is about 65 shore A. In one embodiment, the tubular member 114 comprises material having durometers of above about 50 Shore A with the hard tubular member 114 further having an at least about 10-20 Shore A greater durometer than the associated semi-hard sealing member 113. This correlation enables deformation to occur preferentially in the semi-hard sealing member 113 for any given set of durometers.

The female luer portion body 130 and the inner male portion 111 have been described as being rigid components. The female luer portion body 130 and the tubular member 114 may comprise a material such as, but not limited to, polycarbonate, polypropylene, cyclic olefin copolymer, nylon, glass, and metal. In one embodiment, the female luer portion body 130 and the tubular member 114 have a durometer above about 50 Shore A.

In one embodiment, the sealing member 113 and the tubular member 114 are separate components that engage each other. Although any of the various means known in the art may be used, one non-limiting example is the use of an adhesion area on the second end of the inner core. The adhesion area is disposed about at least a portion of the second end 120 of the inner male portion 111 and comprises a feature such as, but not limited to, texturing, cross linking, adhesives, molded surface features, and combinations thereof for aiding adherence of the sealing member to the tubular member 114.

In an exemplary embodiment, the chamber 133 and the inner male portion 111 have corresponding shapes such as to provide for a close fit. In addition, the chamber 133 may have a taper shape such as frustro-conical shape with the base of the cone being the aperture 135 and the top being the transition from the chamber 133 to the regular size of the female fluid flow path 107.

The luer lock 101 described herein may be used to form a connection in a multitude of applications. In one embodiment, the luer lock 101 serves as a connection between a needle and a syringe, with one having the female luer portion 105 and the other having the male luer portion 103. In a preferred embodiment, the male luer portion 103, which has the sealing member 113, is integrated with the syringe; and the female luer portion 105 is integrated with a needle 173, such as a needle hub 172.

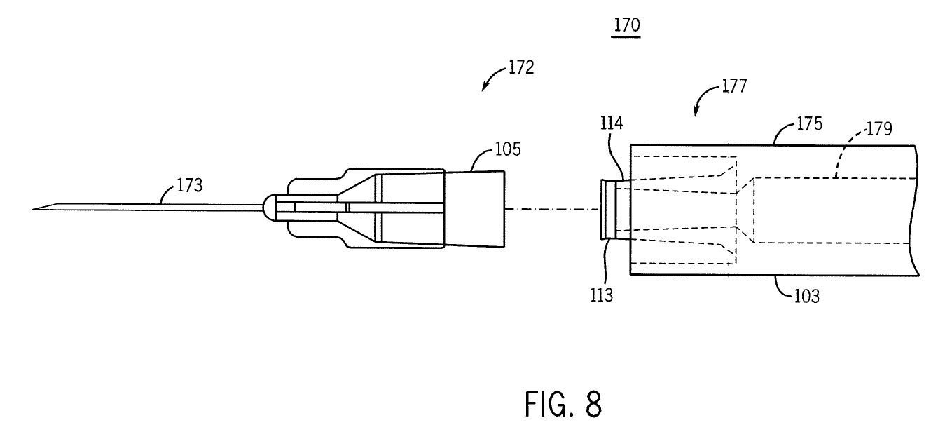

FIG. 8 illustrates one application of the luer lock of the present invention. In the embodiment of FIG. 8, the luer lock 101 is a portion of an injection device 170 comprising a needle assembly 172 and a syringe 177. The syringe includes a syringe body 175 and a corresponding plunger 179 for forcing material out of the syringe 177. The syringe includes, at an end opposite the plunger 179, one of either the male luer portion 103 or female luer portion 105 of the luer lock 101. The needle assembly 172 includes a needle 173 and a portion of a luer lock 101. In one embodiment, the needle assembly 172 includes the male luer portion 103, and in another embodiment the female luer portion 105 is included. It will be appreciated that the syringe 177 will have the corresponding portion of the luer lock 101 such that the syringe 177 and needle 173 can be coupled to form the continuous fluid flow path 108. In a preferred embodiment, the male luer portion 103 is integral to the syringe 177 such that needle assemblies 172 including the female luer portion 105 may be interchangeably used with the syringe 177.

### EXAMPLES

Prototypes were constructed of a polycarbonate male luer with an overmolded 65-durometer thermoplastic elastomer on the tip. The male luer was built according to ISO standard 594/1 with the exception of the second end (distal tip). At the second end, a ring of polycarbonate 0.040″ long×0.015″ thick was replaced by the sealing member 113.

The following has been shown through testing:
A polycarbonate male luer with an overmolded 65-durometer thermoplastic elastomer on the tip can create a 1674 psi seal at 250° or greater rotation when tightened to a nylon female luer. A polycarbonate male luer without this overmolded section creates a 1674 psi seal at 270° or greater rotation when tightened to a female luer. For the purposes of this testing, the tightness was referenced to landmarks (0°)on the chosen luer connections.
A polycarbonate male luer with an overmolded 65-durometer thermoplastic elastomer on the tip can create a seal at a minimum of 1674 psi when connected to nylon, acrylic, and polycarbonate female luers. Repeated use of this male luer seal with different female luers (sealing and unsealing) is possible while still maintaining the seal at the tested PSI.

The foregoing description of embodiments of the present invention have been presented for purposes of illustration and description. It is not intended to be exhaustive or to limit the present invention to the precise form disclosed, and modifications and variations are possible in light of the above teachings or may be acquired from practice of the present invention. The embodiments were chosen and described in order to explain the principles of the present invention and its practical application to enable one skilled in the art to utilize the present invention in various embodiments, and with various modifications, as are suited to the particular use contemplated.

What is claimed is:

1. A luer lock assembly comprising:
a male luer member having a hard tubular member including a first end and a second end and further including a fluid passage therethrough and a semi hard sealing member disposed about at least a portion of the tubular member;
the semi-hard sealing member comprising material having durometers of about 10-100 Shore A and the hard tubular member comprising material having durometers of above about 50 Shore A and at least about 10-20 Shore A greater than the semi-hard sealing member;
a female luer member including a first and second end and further including a body with a hollow passage therethrough; and
the first end of the female luer member comprising a chamber integral with the fluid passage and adapted to receive the male luer member;
wherein when the second end of the tubular member is inserted into a first end of the female luer member, the sealing member engages the chamber and provides a seal between the chamber and the tubular member forming a continuous fluid flow passage.

2. The luer lock assembly of claim 1, wherein the semi-hard sealing member comprises material having durometers of about 30-80 Shore A.

3. The luer lock assembly of claim 1, wherein the semi-hard sealing member comprises material having a durometer of about 65 shore A.

4. The luer lock assembly of claim 1, wherein the tubular member and the hard female luer body comprise a material selected from the group consisting of polycarbonate, polypropylene, cyclic olefin copolymer, nylon, glass, and metal.

5. The luer lock assembly of claim 1 further comprising an adhesion area disposed about at least a portion of the tubular member and comprising an adherence feature selected from the group consisting of texturing, cross linking, adhesives, molded surface features, and combinations thereof for aiding adherence of the sealing member to the tubular member.

6. The luer lock assembly of claim 1, wherein the sealing member comprises at least one rib or a textured surface about its circumference.

7. The luer lock assembly of claim 1, wherein a diameter of the first end of the hard tubular member is greater than the diameter of the second end of the hard male luer member inner core such that the tubular member is tapered.

8. The luer lock assembly of claim 1, wherein the sealing member comprises thermoset or thermoplastic elastomers and has a variable thickness.

9. The luer lock assembly of claim 8, wherein the sealing member comprises material selected from the group consisting of silicone, polyurethane, polyethylene, nylon, polyester, polysulfone, and combinations thereof

10. The luer lock assembly of claim 1, further comprising a needle affixed to the first end of the male luer member and a syringe affixed to the second end of the female luer member, a continuous fluid flow path formed from the needle through the male luer member and female luer member to the syringe.

11. The luer lock assembly of claim 1, further comprising a syringe affixed to the first end of the male luer member and a needle affixed to the second end of the female luer member, a continuous fluid flow path formed from the needle through the female luer member and male luer member to the syringe.

12. A syringe assembly comprising:
a needle having a fluid flow path therethrough, the needle affixed to a first end of a needle connection joint;
the needle connection joint including a fluid flow path therethrough and connected at a second end to a fluid container;
the needle connection joint comprising a first portion and a second portion, the first portion including a protrusion;
a seal circumscribed about at least a portion of the protrusion and the second portion having an opening for receiving the protrusion, the seal comprising material having durometers of between about 10 and 100 Shore A; and
the first portion and second portion including a structure such that when connected a continuous fluid flow path is formed there through, with the protrusion partially disposed within the second portion through the opening and sealed by interaction of the seal against an inner wall of the second portion.

13. The syringe assembly of claim 12, wherein the first portion includes a first end and a second end, the protrusion comprising the second end and the first end attached to the needle and further wherein the second portion includes a first end and a second end, the opening positioned at the first end and the second end attached to the fluid container.

14. The syringe assembly of claim 12, wherein the first portion includes a first end and a second end, the protrusion comprising the second end and the first end attached to the fluid container and further wherein the second portion includes a first end and a second end, the opening positioned at the first end and the second end attached to the needle.

15. The syringe assembly of claim 12, wherein the seal comprises a material having durometers of about 30-80 Shore A.

16. The syringe assembly of claim 12, wherein the first portion and the second portion comprise a material selected from the group consisting of polycarbonate, polypropylene, cyclic olefin copolymer, nylon, glass, and metal.

17. The syringe assembly of claim 12 further comprising an adhesion area on the first portion the adhesion area disposed about at least a portion of the protrusion and comprising a feature selected from the group consisting of texturing, cross linking, adhesives, molded surface features, and combinations thereof for aiding adherence of the sealing member to the inner core.

18. The syringe assembly of claim 12, wherein the sealing member includes an outer surface and an inner surface, the inner surface contacting the protrusion and the outer surface of the sealing member having at least one rib or a textured surface.

19. The syringe assembly of claim 12, wherein the sealing member circumscribes substantially the entire protrusion.

20. The syringe assembly of claim 12 wherein the sealing member comprises thermoset or thermoplastic elastomers and has a variable thickness.

21. A luer lock assembly comprising:
a male luer member having a hard tubular member including a first end and a second end and further including a fluid passage therethrough;
a female luer member including a first and second end and further including a hollow rigid body with a fluid passage therethrough;
a semi hard sealing member disposed about at least an interior portion of the hollow rigid body and forming at least a portion of the female luer member fluid passage, the semi-hard sealing member comprising material having durometers of about 10-100 Shore A and the hollow rigid body and the male hard tubular member comprising material including an associated durometer of above about 50 Shore A and at least about 10-20 Shore A greater than the semi-hard sealing member
the first end of the female luer member comprising a chamber integral with the fluid passage and adapted to receive the male luer member, the semi-hard sealing member disposed about an interior of the chamber;
wherein when the second end of the tubular member is inserted into a first end of the female luer member, the sealing member engages the tubular member and provides a seal between the chamber and the tubular member forming a continuous hollow passage.

22. The luer lock assembly of claim 21, wherein the semi-hard sealing member comprises material having durometers of about 30-80 Shore A.

23. The luer lock assembly of claim 21, wherein the semi-hard sealing member comprises material having a durometer of about 65 shore A.

24. The luer lock assembly of claim 21, wherein the tubular member and the hard female luer body comprise a material selected from the group consisting of polycarbonate, polypropylene, cyclic olefin copolymer, nylon, glass, and metal.

25. The luer lock assembly of claim 21 wherein the sealing member comprises thermoplastic or thermoset elastomers.
